(19) United States
(12) Patent Application Publication
(10) Pub. No.: US 2011/0200643 A1
(43) Pub. Date: Aug. 18, 2011

(54) EMULSIONS FOR PRODUCING MEDICINAL PRODUCTS

(75) Inventor: Regis Cartier, Wiesbaden (DE)

(73) Assignee: BOEHRINGER INGELHEIM INTERNATIONAL GMBH, Ingelheim am Rhein (DE)

(21) Appl. No.: 12/996,702

(22) PCT Filed: Jun. 8, 2009
(86) PCT No.: PCT/EP2009/057009

§ 371 Date: Mar. 28, 2011

(30) Foreign Application Priority Data
Jun. 9, 2008 (EP) 08104313.5

(51) Int. Cl. A61K 9/107; A61K 31/58; A61K 31/137; A61P 11/06; A61P 11/00

(52) U.S. Cl. 424/400; 514/174; 514/653

(57) ABSTRACT

The invention relates to the preparation of stable, polymer-based emulsions which have an ionic strength of more than 10 mM in their aqueous phase, and to processes for preparing them and medicaments that can be obtained using these emulsions.

The invention relates to processes for preparing stable polymer-based emulsions as well as the medicaments that can be obtained using these processes. In one particular embodiment, the invention relates to stable polymer-based emulsions for pulmonary or nasal inhalation and the use of these medicaments for the treatment of respiratory complaints, particularly for the treatment of COPD (chronic obstructive pulmonary disease) and asthma.

### BACKGROUND TO THE INVENTION

By emulsions are meant systems in which a dispersed phase (oil or water, depending on whether it is a W/O or O/W emulsion) with a droplet diameter usually in the nanometre to micrometre range is present. This high degree of division (i.e. the very small droplets) produces a very large surface area on the inner phase. Because of the consequent high interface energies this would lead to immediate destabilisation, which would necessarily result in a coalescence of the small droplets. However, an essential characteristic of emulsions is that they are thermodynamically stable and form a clear to opalescent, thin liquid system of an oily consistency.

The special properties of the emulsions are closely related to their colloidal chemical structure which is a consequence of the chemical composition and also the method of producing the emulsions.

In general so-called emulsifiers are used as adjuvants for preparing emulsions. They have the functions of:
reducing the surface tension between the non-miscible liquids
stabilising the droplets in the emulsion by steric effects and/or electrostatic repulsion forces.

### AIM OF THE INVENTION

Conventional emulsifiers for preparing W/O (water in oil) emulsions consist chemically of a water-soluble and a lipophilic fraction within the molecular structure. In the prior art lipids and polymers are predominantly used.

The pharmaceutical performance of a medicament which contains an emulsion can be improved by minimising the hydrophilic fraction of an emulsifier. By the pharmaceutical performance are meant the physical properties, e.g. the stability, the physicochemical properties, pharmacological properties, toxicological properties and pharmacokinetic properties. These properties can be observed inter alia in the maintenance of a high glass transition temperature if the emulsion is used as a precursor of solid medicaments, a high load rate of an active substance in the emulsion, an improvement in a controlled release of an active substance over time, a reduction in the toxicity or a delayed breakdown of the formulation over time.

The aim of the present invention is thus to provide stable emulsions having improved emulsifiers and processes for preparing them. In another aspect the invention sets out to provide emulsions having biocompatible adjuvants. A particular aim of the invention is to prepare stable emulsions with weak emulsifiers and processes for preparing them. The invention also sets out to provide medicaments which contain the emulsions according to the invention.

### DETAILED DESCRIPTION OF THE INVENTION

It has been found that, surprisingly, the W/O (water in oil) emulsions according to the invention solve the problems set out hereinbefore if they are prepared using an aqueous phase which contains a dilute saline solution.

W/O (water in oil) emulsions according to the invention are also characterised in that the ionic strength of the aqueous phase that is used for preparing the emulsions according to the invention has an ionic strength of more than 10 mM (unit: millimole/litre). An ionic strength of 15 mM, 20 mM, 30 mM and 50 mM is preferred.

By ionic strength/is meant

$$I = \frac{1}{2} {\sum }_{i = 1}^{n} {c}_{i} {z}_{i}^{2}$$

wherein
n=the number of types of ion
c=the molar concentration of the particular type of ion
z=the charge (valency) of the individual Ions.

Within the scope of the invention W/O (water in oil) emulsions according to the invention may be prepared by combining the aqueous phase, before the preparation of the emulsion, with an alkali metal salt, an alkaline earth metal salt, an acid addition salt of an active substance or a combination thereof. This may be done by dissolving a salt, for example, in the aqueous phase. This salt may be an inorganic salt or an organic salt, for example the acid addition salt of an active substance. Examples of possible salts are the alkali metal salts and alkaline earth metal salts, while the anion may be selected from among the fluoride, chloride, bromide, iodide, sulphate, phosphate, methanesulphonate, nitrate, maleate, acetate, citrate, fumarate, tartrate, oxalate, succinate, benzoate and p-toluenesulphonate. The anion may also be an anion with a single negative charge, selected from among the fluoride, chloride, bromide, methanesulphonate and p-toluenesulphonate, particularly preferably bromide and chloride.

The cation may be selected for example from among lithium, sodium, potassium, beryllium, magnesium, calcium, strontium and barium. Of particular importance are sodium, potassium, magnesium and calcium.

Also of particular importance is sodium chloride.

According to the invention the W/O (water in oil) emulsions in a specific embodiment are characterised in that the droplets of emulsion have a hydrodynamic diameter of between 200 nm and 3000 nm, preferably 300 nm and 2500 nm, preferably 500 nm and 2500 nm, preferably 500 nm and 2100 nm. By the hydrodynamic diameter is meant the mean equivalent diameter such as may be determined using photon correlation spectroscopy (more details in the Experimental Section).

Preferred emulsifiers according to the invention are selected from among the polymers. For example, emulsifiers selected from the PEG-[lactide-co-glycolide] category are suitable for preparing the W/O (water in oil) emulsions according to the invention. Such substances are known by the name Resomer® (Boehringer Ingelheim Pharma GmbH & Co. KG, Germany). Polymers from this category of substances are particularly suitable when they have:
a PEG content of 1-15%,
a molecular mass of between 37.5-600 kDa,
a diblock structure or triblock structure,
a glycolide content of 0-50%
and a D-lactide content of 0-25%.

By a diblock structure or triblock structure is meant that the polymer is made up of different units which are regularly repeated at a molecular level.

For example, emulsifiers are selected from among the block copolymers. These contain at least one water-soluble block (block B) and at least one non-water-soluble block (block A).

The structure of the block copolymer may be for example a diblock structure (A-B) or a triblock structure (A-B-A).

PEG (polyethyleneglycol) is used in particular as a water-soluble block. A polyester compound is used in particular as a non-water-soluble block. For example, the category of poly-(lactide-co-glycolide) polymers is used as the polyester block.

The PEG-[poly-(lactide-co-glycolides] listed in Table 1 are of particular importance as emulsifiers for W/O (water in oil) emulsions according to the invention.

TABLE 1
Polymers used for the preparation of emulsions (manufacturer: Boehringer Ingelheim); M_{w} =
molecular weight in [kD] (calculated theoretically according to the manufacturing process on
the basis of the quantities of monomer used); T_{g} = glass transition temperature in [° C.].

| Name | Structure | (weight or molar) Composition | M_{w} | T_{g} |
|---|---|---|---|---|
| RGP d 5055 | diblock | 5 kDa-PEG - [D,L-lactide/glycolide] | 100 | 39 |
| | | 5%/47.5%/47.5% | | |
| LRP d 7055 | diblock | 5 kDa-PEG - [D-lactide/L-lactide] | 100 | 41 |
| | | 5%/15.2%/79.8% | | |
| RGP d 50105 | diblock | 5 kDa-PEG - [D,L-lactide/glycolide] | 50 | 32 |
| | | 10%/43.5%/46.5% | | |
| LGP d 8555 | diblock | 5 kDa-PEG - [L-lactide/glycolide] | 100 | 44 |
| | | 5%/77.9%/17.1% | | |
| LRP t 7046 | triblock | [D-lactide/L-lactide] - 6 kDa-PEG - [D-lactide/L-lactide] | 150 | 49 |
| | | 7.68%/40.32%/4%/7.68%/40.32% | | |
| LRP t 7016 | triblock | [D-lactide/L-lactide] - 6 kDa-PEG - [D-lactide/L-lactide] | 600 | n.d. |
| | | 6.93%/42.57%/1%/6.93%/42.57% | | |
| LP t 156 | triblock | L-lactide - 6 kDa-PEG - L-lactide | 37.5 | 31 |
| | | 42%/16%/42% | | |
| LGP t 8546 | triblock | [L-lactide/glycolide] - 6 kDa-PEG - [L-lactide/glycolide] | 150 | 45 |
| | | 39.36%/8.64%/4%/39.36%/8.64% | | |
| LGP t 8516 | triblock | [L-lactide/glycolide] - 6 kDa-PEG - [L-lactide/glycolide] | 600 | n.d. |
| | | 41.09%/8.4%/4%/41.09%/8.4% | | |
| LP t 52 | triblock | L-lactide - 2 kDa-PEG - L-lactide | 40 | n.d. |
| | | 47.5%/5%/47.5% | | |
| LGP t 5046 | triblock | [L-lactide/glycolide] - 6 kDa-PEG - [L-lactide/glycolide] | 150 | 43 |
| | | 23.52%/24.48%/4%/23.52%/24.48% | | |
| LGP t 50156 | triblock | [L-lactide/glycolide] - 6 kDa-PEG - [L-lactide/glycolide] | 40 | 25 |
| | | 21.25%/21.25%/15%/21.25%/21.25% | | |

Moreover the invention relates to processes with which the problems according to the invention may be solved. Surprisingly, W/O (water in oil) emulsions according to the invention may be prepared by adding an emulsifier selected from among the PEG-[lactide-co-glycolide] category of substances to an organic phase. In another step, a salt is dissolved in the aqueous phase. This salt may be an inorganic salt or an organic salt, for example the acid addition salt of an active substance. Examples of possible salts are the alkali metal salts and alkaline earth metal salts, preferably sodium chloride. It is also possible to dissolve a combination of salts in the aqueous phase.

According to the invention, equivalent to the use of the salts is the use of active substances which on the basis of their ion structure also have an ionic strength of more than 10 mM, also preferably 15 mM, 20 mM, 30 mM and 50 mM.

The emulsion may be obtained by homogenising the organic and the aqueous phase by intensive mixing. The mixing process can thus be carried out in an ultrasound bath or an ultrasound finger with the simultaneous action of ultrasound on the mixture.

The invention further relates to the use of the W/O (water in oil) emulsions for preparing dry powder formulations for medicaments that are to be administered by inhalation. The invention also relates to the use of the emulsions according to the invention as spray solutions for spray drying processes. The spray-drying of pure active substances for inhalation purposes (powder inhalation) is also described in the prior art [e.g.: EP 0 072 046 A1; WO 2000 000176 A1; U.S. Pat. No. 6,019,968; A. Chawla, K. M. G. Taylor, J. M. Newton, M. C. R. Johnson, Int. J. Pharm, 108 (3), (1994), 233-240].

According to the invention the W/O (water in oil) emulsions thus obtained may be used for preparing a medicament. They are preferably used to prepare a medicament for treating respiratory complaints, particularly for treating COPD and/or asthma. The invention also relates to the use of the W/O (water in oil) emulsions thus obtained for preparing a medicament for use by inhalation, particularly for preparing a medicament for inhalation which allows a delayed release of the active substance.

The chemical compounds listed hereinafter (active substances) may be used on their own or in combination as the medicament-relevant component of the W/O (water in oil) emulsions according to the invention, preferably as a component of the aqueous phase.

In the compounds mentioned below, W is a pharmacologically active substance and is selected (for example) from among the betamimetics, anticholinergics, corticosteroids, PDE4-inhibitors, LTD4-antagonists, EGFR-inhibitors, dopamine agonists, H1-antihistamines, PAF-antagonists and Pl3-kinase inhibitors. Moreover, double or triple combinations of W may be combined and used in the device according to the invention. Combinations of W might be, for example:
W denotes a betamimetic, combined with an anticholinergic, corticosteroid, PDE4-inhibitor, EGFR-inhibitor or LTD4-antagonist,
W denotes an anticholinergic, combined with a betamimetic, corticosteroid, PDE4-inhibitor, EGFR-inhibitor or LTD4-antagonist,
W denotes a corticosteroid, combined with a PDE4-inhibitor, EGFR-inhibitor or LTD4-antagonist
W denotes a PDE4-inhibitor, combined with an EGFR-inhibitor or LTD4-antagonist
W denotes an EGFR-inhibitor, combined with an LTD4-antagonist.

The compounds used as betamimetics are preferably compounds selected from among albuterol, arformoterol, bambuterol, bitolterol, broxaterol, carbuterol, clenbuterol, fenoterol, formoterol, hexoprenaline, ibuterol, isoetharine, isoprenaline, levosalbutamol, mabuterol, meluadrine, metaproterenol, orciprenaline, pirbuterol, procaterol, reproterol, rimiterol, ritodrine, salmefamol, salmeterol, soterenol, sulphonterol, terbutaline, tiaramide, tolubuterol, zinterol, CHF-1035, HOKU-81, KUL-1248 and
3-(4-{6-[2-hydroxy-2-(4-hydroxy-3-hydroxymethyl-phenyl)-ethylamino]-hexyloxy}-butyl)-benzyl-sulphonamide
5-[2-(5,6-diethyl-indan-2-ylamino)-1-hydroxy-ethyl]-8-hydroxy-1H-quinolin-2-one
4-hydroxy-7-[2-{[2-{[3-(2-phenylethoxy)propyl]sulphonyl}ethyl]-amino}ethyl]-2(3H)-benzothiazolone
1-(2-fluoro-4-hydroxyphenyl)-2-[4-(1-benzimidazolyl)-2-methyl-2-butylamino]ethanol
1-[3-(4-methoxybenzyl-amino)-4-hydroxyphenyl]-2-[4-(1-benzimidazolyl)-2-methyl-2-butylamino]ethanol
1-[2H-5-hydroxy-3-oxo-4H-1,4-benzoxazin-8-yl]-2-[3-(4-N,N-dimethylaminophenyl)-2-methyl-2-propylamino]ethanol
1-[2H-5-hydroxy-3-oxo-4H-1,4-benzoxazin-8-yl]-2-[3-(4-methoxyphenyl)-2-methyl-2-propylamino]ethanol
1-[2H-5-hydroxy-3-oxo-4H-1,4-benzoxazin-8-yl]-2-[3-(4-n-butyloxyphenyl)-2-methyl-2-propylamino]ethanol
1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol
5-hydroxy-8-(1-hydroxy-2-isopropylaminobutyl)-2H-1,4-benzoxazin-3-(4H)-one
1-(4-amino-3-chloro-5-trifluoromethylphenyl)-2-tert.-butylamino)ethanol
6-hydroxy-8-{1-hydroxy-2-[2-(4-methoxy-phenyl)-1,1-dimethyl-ethylamino]-ethyl}-4H-benzo[1,4]oxazin-3-one
6-hydroxy-8-{1-hydroxy-2-[2-(ethyl 4-phenoxy-acetate)-1,1-dimethyl-ethylamino]-ethyl}-4H-benzo[1,4]oxazin-3-one
6-hydroxy-8-{1-hydroxy-2-[2-(4-phenoxy-acetic acid)-1,1-dimethyl-ethylamino]-ethyl}-4H-benzo[1,4]oxazin-3-one
8-{2-[1,1-dimethyl-2-(2,4,6-trimethylphenyl)-ethylamino]-1-hydroxy-ethyl}-6-hydroxy-4H-benzo[1,4]oxazin-3-one
6-hydroxy-8-{1-hydroxy-2-[2-(4-hydroxy-phenyl)-1,1-dimethyl-ethylamino]-ethyl}-4H-benzo[1,4]oxazin-3-one
6-hydroxy-8-{1-hydroxy-2-[2-(4-isopropyl-phenyl)-1.1dimethyl-ethylamino]-ethyl}-4H-benzo[1,4]oxazin-3-one
8-{2-[2-(4-ethyl-phenyl)-1,1-dimethyl-ethylamino]-1-hydroxy-ethyl}-6-hydroxy-4H-benzo[1,4]oxazin-3-one
8-{2-[2-(4-ethoxy-phenyl)-1,1-dimethyl-ethylamino]-1-hydroxy-ethyl}-6-hydroxy-4H-benzo[1,4]oxazin-3-one
4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butyric acid
8-{2-[2-(3,4-difluoro-phenyl)-1,1-dimethyl-ethylamino]-1-hydroxy-ethyl}-6-hydroxy-4H-benzo[1,4]oxazin-3-one
1-(4-ethoxy-carbonylamino-3-cyano-5-fluorophenyl)-2-(tert-butylamino)ethanol
2-hydroxy-5-(1-hydroxy-2-{2-[4-(2-hydroxy-2-phenyl-ethylamino)-phenyl]-ethylamino}-ethyl)-benzaldehyde
N-[2-hydroxy-5-(1-hydroxy-2-{2-[4-(2-hydroxy-2-phenyl-ethylamino)-phenyl]-ethylamino}-ethyl)-phenyl]-formamide
8-hydroxy-5-(1-hydroxy-2-{2-[4-(6-methoxy-biphenyl-3-ylamino)-phenyl]-ethylamino}-ethyl)-1H-quinolin-2-one
8-hydroxy-5-[1-hydroxy-2-(6-phenethylamino-hexylamino)-ethyl]-1H-quinolin-2-one
5-[2-(2-{4-[4-(2-amino-2-methyl-propoxy)-phenylamino]-phenyl}-ethylamino)-1-hydroxy-ethyl]-8-hydroxy-1H-quinolin-2-one
[3-(4-{6-[2-hydroxy-2-(4-hydroxy-3-hydroxymethyl-phenyl)-ethylamino]-hexyloxy}-butyl)-5-methyl-phenyl]-urea
4-(2-{6-[2-(2,6-dichloro-benzyloxy)-ethoxy]-hexylamino}-1-hydroxy-ethyl)-2-hydroxymethyl-phenol
3-(4-{6-[2-hydroxy-2-(4-hydroxy-3-hydroxymethyl-phenyl)-ethylamino]-hexyloxy}-butyl)-benzylsulphonamide
3-(3-{7-[2-hydroxy-2-(4-hydroxy-3-hydroxymethyl-phenyl)-ethylamino]-heptyloxy}-propyl)-benzylsulphonamide
4-(2-{6-[4-(3-cyclopentanesulphonyl-phenyl)-butoxy]-hexylamino}-1-hydroxy-ethyl)-2-hydroxymethyl-phenol
N-adamantan-2-yl-2-(3-{2-[2-hydroxy-2-(4-hydroxy-3-hydroxymethyl-phenyl)-ethylamino]-propyl}-phenyl)-acetamide
   optionally in the form of the racemates, enantiomers, diastereomers thereof and optionally in the form of the pharmacologically acceptable acid addition salts, solvates or hydrates thereof. According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The anticholinergics used are preferably compounds selected from among the tiotropium salts, preferably the bromide salt, oxitropium salts, preferably the bromide salt, flutropium salts, preferably the bromide salt, ipratropium salts, preferably the bromide salt, glycopyrronium salts, preferably the bromide salt, trospium salts, preferably the chloride salt, tolterodine. In the above-mentioned salts the cations are the pharmacologically active constituents. As anions the above-mentioned salts may preferably contain the chloride, bromide, iodide, sulphate, phosphate, methanesulphonate, nitrate, maleate, acetate, citrate, fumarate, tartrate, oxalate, succinate, benzoate or p-toluenesulphonate, while chloride, bromide, iodide, sulphate, methanesulphonate or p-toluenesulphonate are preferred as counter-ions. Of all the salts the chlorides, bromides, iodides and methanesulphonates are particularly preferred.

Other preferred anticholinergics are selected from among the salts of formula AC-1

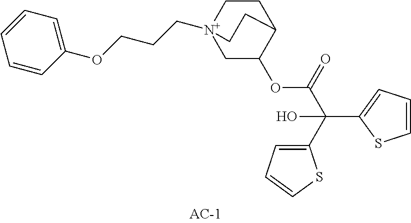

wherein X⁻ denotes an anion with a single negative charge, preferably an anion selected from among the fluoride, chloride, bromide, iodide, sulphate, phosphate, methanesulphonate, nitrate, maleate, acetate, citrate, fumarate, tartrate, oxalate, succinate, benzoate and p-toluenesulphonate, preferably an anion with a single negative charge, particularly preferably an anion selected from among the fluoride, chloride, bromide, methanesulphonate and p-toluenesulphonate, particularly preferably bromide, optionally in the form of the racemates, enantiomers or hydrates thereof. Of particular importance are those pharmaceutical combinations which contain the enantiomers of formula AC-1-ene

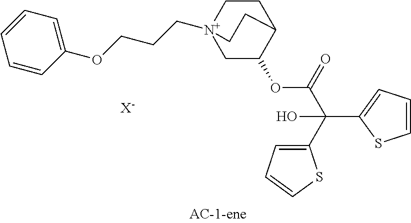

wherein X⁻ may have the above-mentioned meanings. Other preferred anticholinergics are selected from the salts of formula AC-2

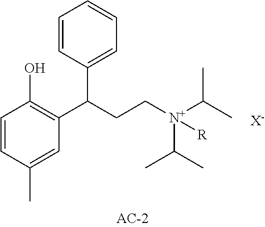

wherein R denotes either methyl or ethyl and wherein X⁻ may have the above-mentioned meanings. In an alternative embodiment the compound of formula AC-2 may also be present in the form of the free base AC-2-base.

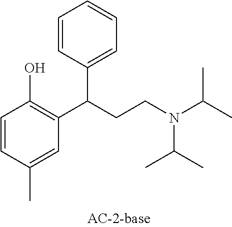

Other specified compounds are:
tropenol 2,2-diphenylpropionate methobromide
scopine 2,2-diphenylpropionate methobromide
scopine 2-fluoro-2,2-diphenylacetate methobromide
tropenol 2-fluoro-2,2-diphenylacetate methobromide
tropenol 3,3′,4,4′-tetrafluorobenzilate methobromide
scopine 3,3′,4,4′-tetrafluorobenzilate methobromide
tropenol 4,4′-difluorobenzilate methobromide
scopine 4,4′-difluorobenzilate methobromide
tropenol 3,3′-difluorobenzilate methobromide
scopine 3,3′-difluorobenzilate methobromide
tropenol 9-hydroxy-fluorene-9-carboxylate methobromide
tropenol 9-fluoro-fluorene-9-carboxylate methobromide
scopine 9-hydroxy-fluorene-9-carboxylate methobromide
scopine 9-fluoro-fluorene-9-carboxylate methobromide
tropenol 9-methyl-fluorene-9-carboxylate methobromide
scopine 9-methyl-fluorene-9-carboxylate methobromide
cyclopropyltropine benzilate methobromide;
cyclopropyltropine 2,2-diphenylpropionate methobromide
cyclopropyltropine 9-hydroxy-xanthene-9-carboxylate methobromide
cyclopropyltropine 9-methyl-fluorene-9-carboxylate methobromide
cyclopropyltropine 9-methyl-xanthene-9-carboxylate methobromide
cyclopropyltropine 9-hydroxy-fluorene-9-carboxylate methobromide
cyclopropyltropine methyl 4,4′-difluorobenzilate methobromide
tropenol 9-hydroxy-xanthene-9-carboxylate methobromide
scopine 9-hydroxy-xanthene-9-carboxylate methobromide
tropenol 9-methyl-xanthene-9-carboxylate methobromide
scopine 9-methyl-xanthene-9-carboxylate methobromide
tropenol 9-ethyl-xanthene-9-carboxylate methobromide
tropenol 9-difluoromethyl-xanthene-9-carboxylate methobromide
scopine 9-hydroxymethyl-xanthene-9-carboxylate methobromide

The above-mentioned compounds may also be used as salts within the scope of the present invention, wherein instead of the methobromide the salts metho-X are used, wherein X may have the meanings given hereinbefore for X⁻.

As corticosteroids it is preferable to use compounds selected from among beclomethasone, betamethasone, budesonide, butixocort, ciclesonide, deflazacort, dexamethasone, etiprednol, flunisolide, fluticasone, loteprednol, mometasone, prednisolone, prednisone, rofleponide, triamcinolone, RPR-106541, NS-126, ST-26 and
(S)-fluoromethyl6,9-difluoro-17-[(2-furanylcarbonyl)oxy]-11-hydroxy-16-methyl-3-oxo-androsta-1,4-diene-17-carbothionate
(S)-(2-oxo-tetrahydro-furan-3S-yl)6,9-difluoro-11-hydroxy-16-methyl-3-oxo-17-propionyloxy-androsta-1,4-diene-17-carbothionate,
cyanomethyl 6α,9α-difluoro-11β-hydroxy-16α-methyl-3-oxo-17α-(2,2,3,3-tertamethylcyclopropylcarbonyl)oxy-androsta-1,4-diene-17β-carboxylate
   optionally in the form of the racemates, enantiomers or diastereomers thereof and optionally in the form of the salts and derivatives thereof, the solvates and/or hydrates thereof. Any reference to steroids includes a reference to any salts or derivatives, hydrates or solvates thereof which may exist. Examples of possible salts and derivatives of the steroids may be: alkali metal salts, such as for example sodium or potassium salts, sulphobenzoates, phosphates, isonicotinates, acetates, dichloroacetates, propionates, dihydrogen phosphates, palmitates, pivalates or furoates.

PDE4-inhibitors which may be used are preferably compounds selected from among enprofyllin, theophyllin, roflumilast, ariflo (cilomilast), tofimilast, pumafentrin, lirimilast, arofyllin, atizoram, D-4418, Bay-198004, BY343, CP-325.366, D-4396 (Sch-351591), AWD-12-281 (GW-842470), NCS-613, CDP-840, D-4418, PD-168787, T-440, T-2585, V-11294A, CI-1018, CDC-801, CDC-3052, D-22888, YM-58997, Z-15370 and
N-(3,5-dichloro-1-oxo-pyridin-4-yl)-4-difluoromethoxy-3-cyclopropylmethoxybenzamide
(−)p-[(4aR*,10bS*)-9-ethoxy-1,2,3,4,4a,10b-hexahydro-8-methoxy-2-methylbenzo[s][1,6]naphthyridin-6-yl]N,N-diisopropylbenzamide
(R)-(+)-1-(4-bromobenzyl)-4-[(3-cyclopentyloxy)-4-methoxyphenyl]-2-pyrrolidone
3-(cyclopentyloxy-4-methoxyphenyl)-1-(4-N′-[N-2-cyano-S-methyl-isothioureido]benzyl)-2-pyrrolidone
cis[4-cyano-4-(3-cyclopentyloxy-4-methoxyphenyl)cyclohexane-1-carboxylic acid]
2-carbomethoxy-4-cyano-4-(3-cyclopropylmethoxy-4-difluoromethoxy-phenyl)cyclohexan-1-one
cis[4-cyano-4-(3-cyclopropylmethoxy-4-difluoromethoxyphenyl)cyclohexan-1-ol]
(R)-(+)-ethyl[4-(3-cyclopentyloxy-4-methoxyphenyl)pyrrolidin-2-ylidene]acetate
(S)-(−)-ethyl[4-(3-cyclopentyloxy-4-methoxyphenyl)pyrrolidin-2-ylidene]acetate
9-cyclopentyl-5,6-dihydro-7-ethyl-3-(2-thienyl)-9H-pyrazolo[3,4-c]-1,2,4-triazolo[4,3-a]pyridine
9-cyclopentyl-5,6-dihydro-7-ethyl-3-(tert-butyl)-9H-pyrazolo[3,4-c]-1,2,4-triazolo[4,3-a]pyridine
   optionally in the form of the racemates, enantiomers or diastereomers thereof and optionally in the form of the pharmacologically acceptable acid addition salts thereof, the solvates and/or hydrates thereof. According to the invention the acid addition salts of the PDE4 inhibitors are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The LTD4-antagonists used are preferably compounds selected from among montelukast, pranlukast, zafirlukast, MCC-847 (ZD-3523), MN-001, MEN-91507 (LM-1507), VUF-5078, VUF-K-8707, L-733321 and
1-((((R)-(3-(2-(6,7-difluoro-2-quinolinyl)ethenyl)phenyl)-3-(2-(2-hydroxy-2-propyl)phenyl)thio)methylcyclopropane-acetic acid,
1-(((1(R)-3(3-(2-(2,3-dichlorothieno[3,2-b]pyridin-5-yl)-(E)-ethenyl)phenyl)-3-(2-(1-hydroxy-1-methylethyl)phenyl)propyl)thio)methyl)cyclopropaneacetic acid
[2-[[2-(4-tert-butyl-2-thiazolyl)-5-benzofuranyl]oxymethyl]phenyl]acetic acid
   optionally in the form of the racemates, enantiomers or diastereomers thereof and optionally in the form of the pharmacologically acceptable acid addition salts, solvates and/or hydrates thereof. According to the invention these acid addition salts are preferably selected from among the hydrochloride, hydrobromide, hydroiodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate. By salts or derivatives which the LTD4-antagonists may optionally be capable of forming are meant, for example: alkali metal salts, such as for example sodium or potassium salts, alkaline earth metal salts, sulphobenzoates, phosphates, isonicotinates, acetates, propionates, dihydrogen phosphates, palm itates, pivalates or furoates.

EGFR-inhibitors which may be used are preferably compounds selected from among cetuximab, trastuzumab, ABX-EGF, Mab ICR-62 and
4-[(3-chloro-4-fluorophenyl)amino]-6-{[4-(morpholin-4-yl)-1-oxo-2-buten-1-yl]amino}-7-cyclopropylmethoxy-quinazoline
4-[(3-chloro-4-fluorophenyl)amino]-6-{[4-(N,N-diethylamino)-1-oxo-2-buten-1-yl]-amino}-7-cyclopropylmethoxy-quinazoline
4-[(3-chloro-4-fluorophenyl)amino]-6-{[4-(N,N-dimethylamino)-1-oxo-2-buten-1-yl]amino}-7-cyclopropylmethoxy-quinazoline
4-[(R)-(1-phenyl-ethyl)amino]-6-{[4-(morpholin-4-yl)-1-oxo-2-buten-1-yl]amino}-7-cyclopentyloxy-quinazoline
4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline
4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline
4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline
4-[(3-chloro-4-fluoro-phenyl)amino]-6-[2-((S)-6-methyl-2-oxo-morpholin-4-yl)-ethoxy]-7-methoxy-quinazoline
4-[(3-chloro-4-fluorophenyl)amino]-6-({[4N-(2-methoxy-ethyl)-N-methyl-amino]-1-oxo-2-buten-1-yl}amino)-7-cyclopropylmethoxy-quinazoline
4-[(3-chloro-4-fluorophenyl)amino]-6-{[4-(N,N-dimethylamino)-1-oxo-2-buten-1-yl]amino}-7-cyclopentyloxy-quinazoline
4-[(R)-(1-phenyl-ethyl)amino]-6-{[4-(N,N-to-(2-methoxy-ethyl)-amino)-1-oxo-2-buten-1-yl]amino}-7-cyclopropylmethoxy-quinazoline
4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline
4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline
4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline
4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline
4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline
4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline
4-[(3-chloro-4-fluorophenyl)amino]-6-{[4-(N-cyclopropyl-N-methyl-amino)-1-oxo-2-buten-1-yl}amino]-7-cyclopentyloxy-quinazoline
4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline
4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline
4-[(3-ethynyl-phenyl)amino]-6.7-to-(2-methoxy-ethoxy)-quinazoline
4-[(3-chloro-4-fluorophenyl)amino]-7-[3-(morpholin-4-yl)-propyloxy]-6-[(vinyl-carbonyl)amino]-quinazoline
4-[(R)-(1-phenyl-ethyl)amino]-6-(4-hydroxy-phenyl)-7H-pyrrolo[2,3-d]pyrimidine
3-cyano-4-[(3-chloro-4-fluorophenyl)amino]-6-{[4-(N,N-dimethylamino)-1-oxo-2-buten-1-yl]amino}-7-ethoxy-quinoline
4-{[3-chloro-4-(3-fluoro-benzyloxy)-phenyl]amino}-6-(5-{[(2-methanesulphonyl-ethyl)amino]methyl}-furan-2-yl)quinazoline
4-[(R)-(1-phenyl-ethyl)amino]-6-{[4-((R)-6-methyl-2-oxo-morpholin-4-yl)-1-oxo-2-buten-1-yl]amino}-7-methoxy-quinazoline
4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline
4-[(3-chloro-4-fluorophenyl)amino]-6-({4-[N,N-to-(2-methoxy-ethyl)-amino]-1-oxo-2-buten-1-yl}amino)-7-[(tetrahydrofuran-2-yl)methoxy]-quinazoline
4-[(3-ethynyl-phenyl)amino]-6-{[4-(5.5-dimethyl-2-oxo-morpholin-4-yl)-1-oxo-2-buten-1-yl]amino}-quinazoline
4-[(3-chloro-4-fluoro-phenyl)amino]-6-[2-(2,2-dimethyl-6-oxo-morpholin-4-yl)-ethoxy]-7-methoxy-quinazoline
4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline
4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline
4-[(3-chloro-4-fluoro-phenyl)amino]-6-{2-[4-(2-oxo-morpholin-4-yl)-piperidin-1-yl]-ethoxy}-7-methoxy-quinazoline
4-[(3-chloro-4-fluoro-phenyl)amino]-6-[1-(tert.-butyloxycarbonyl)-piperidin-4-yloxy]-7-methoxy-quinazoline
4-[(3-chloro-4-fluoro-phenyl)amino]-6-(trans-4-amino-cyclohexan-1-yloxy)-7-methoxy-quinazoline
4-[(3-chloro-4-fluoro-phenyl)amino]-6-(trans-4-methanesulphonylamino-cyclohexan-1-yloxy)-7-methoxy-quinazoline
4-[(3-chloro-4-fluoro-phenyl)amino]-6-(tetrahydropyran-3-yloxy)-7-methoxy-quinazoline
4-[(3-chloro-4-fluoro-phenyl)amino]-6-(1-methyl-piperidin-4-yloxy)-7-methoxy-quinazoline
4-[(3-chloro-4-fluoro-phenyl)amino]-6-{1-[(morpholin-4-yl)carbonyl]-piperidin-4-yloxy}-7-methoxy-quinazoline
4-[(3-chloro-4-fluoro-phenyl)amino]-6-{1-[(methoxymethyl)carbonyl]piperidin-4-yloxy}-7-methoxy-quinazoline
4-[(3-chloro-4-fluoro-phenyl)amino]-6-(piperidin-3-yloxy)-7-methoxy-quinazoline
4-[(3-chloro-4-fluoro-phenyl)amino]-6-[1-(2-acetylamino-ethyl)-piperidin-4-yloxy]-7-methoxy-quinazoline
4-[(3-chloro-4-fluoro-phenyl)amino]-6-(tetrahydropyran-4-yloxy)-7-ethoxy-quinazoline
4-[(3-chloro-4-fluoro-phenyl)amino]-6-((S)-tetrahydrofuran-3-yloxy)-7-hydroxy-quinazoline
4-[(3-chloro-4-fluoro-phenyl)amino]-6-(tetrahydropyran-4-yloxy)-7-(2-methoxy-ethoxy)-quinazoline
4-[(3-chloro-4-fluoro-phenyl)amino]-6-{trans-4-[(dimethylamino)sulphonylamino]-cyclohexan-1-yloxy}-7-methoxy-quinazoline
4-[(3-chloro-4-fluoro-phenyl)amino]-6-{trans-4-[(morpholin-4-yl)carbonylamino]-cyclohexan-1-yloxy}-7-methoxy-quinazoline
4-[(3-chloro-4-fluoro-phenyl)amino]-6-{trans-4-[(morpholin-4-yl)sulphonylamino]-cyclohexan-1-yloxy}-7-methoxy-quinazoline
4-[(3-chloro-4-fluoro-phenyl)amino]-6-(tetrahydropyran-4-yloxy)-7-(2-acetylamino-ethoxy)-quinazoline
4-[(3-chloro-4-fluoro-phenyl)amino]-6-(tetrahydropyran-4-yloxy)-7-(2-methanesulphonylamino-ethoxy)-quinazoline
4-[(3-chloro-4-fluoro-phenyl)amino]-6-{1-[(piperidin-1-yl)carbonyl]-piperidin-4-yloxy}-7-methoxy-quinazoline
4-[(3-chloro-4-fluoro-phenyl)amino]-6-(1-aminocarbonylmethyl-piperidin-4-yloxy)-7-methoxy-quinazoline
4-[(3-chloro-4-fluoro-phenyl)amino]-6-(cis-4-{N-[(tetrahydropyran-4-yl)carbonyl]-N-methyl-amino}-cyclohexan-1-yloxy)-7-methoxy-quinazoline
4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline
4-[(3-chloro-4-fluoro-phenyl)amino]-6-(cis-4-{N-[(morpholin-4-yl)sulphonyl]-N-methyl-amino}-cyclohexan-1-yloxy)-7-methoxy-quinazoline
4-[(3-chloro-4-fluoro-phenyl)amino]-6-(trans-4-ethanesulphonylamino-cyclohexan-1-yloxy)-7-methoxy-quinazoline
4-[(3-chloro-4-fluoro-phenyl)amino]-6-(1-methanesulphonyl-piperidin-4-yloxy)-7-ethoxy-quinazoline
4-[(3-chloro-4-fluoro-phenyl)amino]-6-(1-methanesulphonyl-piperidin-4-yloxy)-7-(2-methoxy-ethoxy)-quinazoline
4-[(3-chloro-4-fluoro-phenyl)amino]-6-[1-(2-methoxy-acetyl)-piperidin-4-yloxy]-7-(2-methoxy-ethoxy)-quinazoline
4-[(3-chloro-4-fluoro-phenyl)amino]-6-(cis-4-acetylamino-cyclohexan-1-yloxy)-7-methoxy-quinazoline
4-[(3-ethynyl-phenyl)amino]-6-[1-(tert.-butyloxycarbonyl)-piperidin-4-yloxy]-7-methoxy-quinazoline
4-[(3-ethynyl-phenyl)amino]-6-(tetrahydropyran-4-yloxy]-7-methoxy-quinazoline
4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline
4-[(3-chloro-4-fluoro-phenyl)amino]-6-(cis-4-{N-[(4-methyl-piperazin-1-yl)carbonyl]-N-methyl-amino}-cyclohexan-1-yloxy)-7-methoxy-quinazoline
4-[(3-chloro-4-fluoro-phenyl)amino]-6-{cis-4-[(morpholin-4-yl)carbonylamino]-cyclohexan-1-yloxy}-7-methoxy-quinazoline
4-[(3-chloro-4-fluoro-phenyl)amino]-6-{1-[2-(2-oxopyrrolidin-1-yl)ethyl]-piperidin-4-yloxy}-7-methoxy-quinazoline
4-[(3-chloro-4-fluoro-phenyl)amino]-6-{1-[(morpholin-4-yl)carbonyl]-piperidin-4-yloxy}-7-(2-methoxy-ethoxy)-quinazoline
4-[(3-ethynyl-phenyl)amino]-6-(1-acetyl-piperidin-4-yloxy)-7-methoxy-quinazoline
4-[(3-ethynyl-phenyl)amino]-6-(1-methyl-piperidin-4-yloxy)-7-methoxy-quinazoline
4-[(3-ethynyl-phenyl)amino]-6-(1-methanesulphonyl-piperidin-4-yloxy)-7-methoxy-quinazoline
4-[(3-chloro-4-fluoro-phenyl)amino]-6-(1-methyl-piperidin-4-yloxy)-7(2-methoxy-ethoxy)-quinazoline
4-[(3-chloro-4-fluoro-phenyl)amino]-6-(1-isopropyloxycarbonyl-piperidin-4-yloxy)-7-methoxy-quinazoline
4-[(3-chloro-4-fluoro-phenyl)amino]-6-(cis-4-methylamino-cyclohexan-1-yloxy)-7-methoxy-quinazoline
4-[(3-chloro-4-fluoro-phenyl)amino]-6-{cis-4-[N-(2-methoxy-acetyl)-N-methyl-amino]-cyclohexan-1-yloxy}-7-methoxy-quinazoline
4-[(3-ethynyl-phenyl)amino]-6-(piperidin-4-yloxy)-7-methoxy-quinazoline
4-[(3-ethynyl-phenyl)amino]-6-[1-(2-methoxy-acetyl)-piperidin-4-yloxy]-7-methoxy-quinazoline
4-[(3-ethynyl-phenyl)amino]-6-{1-[(morpholin-4-yl)carbonyl]-piperidin-4-yloxy}-7-methoxy-quinazoline
4-[(3-chloro-4-fluoro-phenyl)amino]-6-{1-[(cis-2,6-dimethyl-morpholin-4-yl)carbonyl]-piperidin-4-yloxy}-7-methoxy-quinazoline
4-[(3-chloro-4-fluoro-phenyl)amino]-6-{1-[(2-methyl-morpholin-4-yl)carbonyl]-piperidin-4-yloxy}-7-methoxy-quinazoline
4-[(3-chloro-4-fluoro-phenyl)amino]-6-{1-[(S,S)-(2-oxa-5-aza-bicyclo[2,2,1]hept-5-yl)carbonyl]-piperidin-4-yloxy}-7-methoxy-quinazoline
4-[(3-chloro-4-fluoro-phenyl)amino]-6-{1-[(N-methyl-N-2-methoxyethyl-amino)carbonyl]-piperidin-4-yloxy}-7-methoxy-quinazoline
4-[(3-chloro-4-fluoro-phenyl)amino]-6-(1-ethyl-piperidin-4-yloxy)-7-methoxy-quinazoline
4-[(3-chloro-4-fluoro-phenyl)amino]-6-{1-[(2-methoxyethyl)carbonyl]-piperidin-4-yloxy}-7-methoxy-quinazoline
4-[(3-chloro-4-fluoro-phenyl)amino]-6-{1-[(3-methoxypropyl-amino)-carbonyl]-piperidin-4-yloxy}-7-methoxy-quinazoline
4-[(3-chloro-4-fluoro-phenyl)amino]-6-[cis-4-(N-methanesulphonyl-N-methyl-amino)-cyclohexan-1-yloxy]-7-methoxy-quinazoline
4-[(3-chloro-4-fluoro-phenyl)amino]-6-[cis-4-(N-acetyl-N-methyl-amino)-cyclohexan-1-yloxy]-7-methoxy-quinazoline
4-[(3-chloro-4-fluoro-phenyl)amino]-6-(trans-4-methylamino-cyclohexan-1-yloxy)-7-methoxy-quinazoline
4-[(3-chloro-4-fluoro-phenyl)amino]-6-[trans-4-(N-methanesulphonyl-N-methyl-amino)-cyclohexan-1-yloxy]-7-methoxy-quinazoline
4-[(3-chloro-4-fluoro-phenyl)amino]-6-(trans-4-dimethylamino-cyclohexan-1-yloxy)-7-methoxy-quinazoline
4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline
4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline
4-[(3-chloro-4-fluoro-phenyl)amino]-6-(1-methanesulphonyl-piperidin-4-yloxy)-7-methoxy-quinazoline 4-[(3-chloro-4-fluoro-phenyl)amino]-6-(1-cyano-piperidin-4-yloxy)-7-methoxy-quinazoline
   optionally in the form of the racemates, enantiomers, diastereomers thereof and optionally in the form of the pharmacologically acceptable acid addition salts, solvates or hydrates thereof. According to the invention these acid addition salts are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The dopamine agonists used are preferably compounds selected from among bromocriptine, cabergoline, alpha-dihydroergocryptine, lisuride, pergolide, pram ipexole, roxindole, ropinirole, talipexole, terguride and viozan, optionally in the form of the racemates, enantiomers, diastereomers thereof and optionally in the form of the pharmacologically acceptable acid addition salts, solvates or hydrates thereof. According to the invention these acid addition salts are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydrooxalate, hydrosuccinate, hydrobenzoate and hydro-p-toluenesulphonate.

H1-Antihistamines which may be used are preferably compounds selected from among epinastine, cetirizine, azelastine, fexofenadine, levocabastine, loratadine, mizolastine, ketotifen, emedastine, dimetindene, clemastine, bamipine, cexchlorpheniramine, pheniramine, doxylamine, chlorphenoxamine, dimenhydrinate, diphenhydramine, promethazine, ebastine, desloratidine and meclozine, optionally in the form of the racemates, enantiomers, diastereomers thereof and optionally in the form of the pharmacologically acceptable acid addition salts, solvates or hydrates thereof. According to the invention these acid addition salts are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The pharmaceutically effective substances, formulations or mixtures of substances used may be any inhalable compounds, including also for example inhalable macromolecules, as disclosed in EP 1 003 478. Preferably, substances, formulations or mixtures of substances for treating respiratory complaints which are administered by inhalation are used.

In addition, the compound may come from the group of ergot alkaloid derivatives, the triptans, the CGRP-inhibitors, the phosphodiesterase-V inhibitors, optionally in the form of the racemates, enantiomers or diastereomers thereof, optionally in the form of the pharmacologically acceptable acid addition salts, the solvates and/or hydrates thereof.

Examples of ergot alkaloid derivatives are dihydroergotamine and ergotamine.

### Experimental Section

### (1) MEASURING PROCESS

### Photon Correlation Spectroscopy (Zetasizer, Malvern)

Measuring apparatus: Zetasizer, Malvern, type Zetasizer Nano ZS

### Software: Dispersion Technology Software Version 4.10 (Malvern)

Measuring conditions/measuring parameters—Method:
Measuring process according to the instructions provided by equipment manufacturer. The apparatus calculates the hydrodynamic diameter (Dh) of a suspension and gives the size distribution. The results of the measurements listed below correspond to the main peaks of the size distributions determined.

### (2) EXAMPLES

The emulsion solutions were prepared in the Examples 1 to 4 that follow, such that the active substances or polymers were present dissolved in their respective phase in a concentration of 0.75% (by weight). The phases were combined and treated using an ultrasonic finger for 2 min at 30% power. The tip is immersed 0.5-2 cm in the solution and the ultrasound apparatus is operated at 30%.

### Influence of the Ionic Strength on the Stability of the W/O (Water in Oil) Emulsions According to the Invention

### Example 1

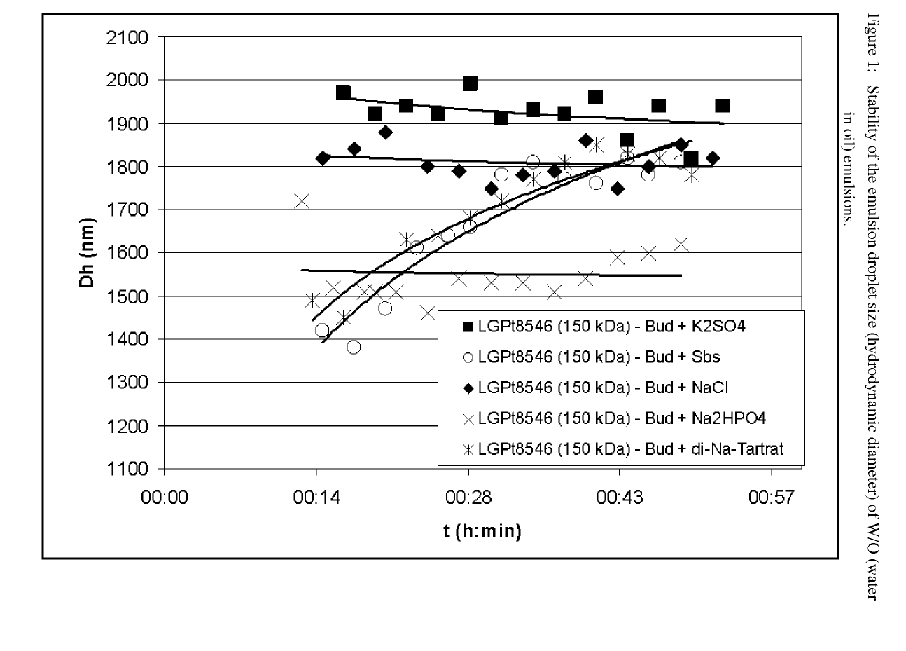

The influence of the salt concentration on the stability and suitability of W/O (water in oil) emulsions was investigated by testing the influence on the stability of different salts at the same ionic strength (20 mM) as the component of the W/O (water in oil) emulsions. FIG. 1 shows W/O (water in oil) emulsions according to the invention which contain as emulsifiers various polymers of the Resomer® type (see the captions to the Figures and the information according to Table 1). Budesonide was dissolved in dichloromethane (DCM), which constitutes the organic phase. The organic phase was then processed with an aqueous phase in the ratio 1:4 (W/O water/DCM emulsion), which contained various salts with an ionic strength of 20 mM, to form an emulsion.

As can be seen from FIG. 1, a stable emulsion can be prepared with all the salts tested, as the emulsion droplet size (hydrodynamic diameter of the emulsion) is in a suitable range between 300 nm and 3000 nm (K2SO4=potassium sulphate, Sbs=salbutamol sulphate salt, NaCl=sodium chloride, Na2HPO4=sodium hydrogen phosphate, di-Na-tartrate=di-sodium tartrate; Bud=budesonide). In addition, the emulsion droplet size remains stably within this range that is suitable for the droplet size, within the measuring period of 1 hour.

### Example 2

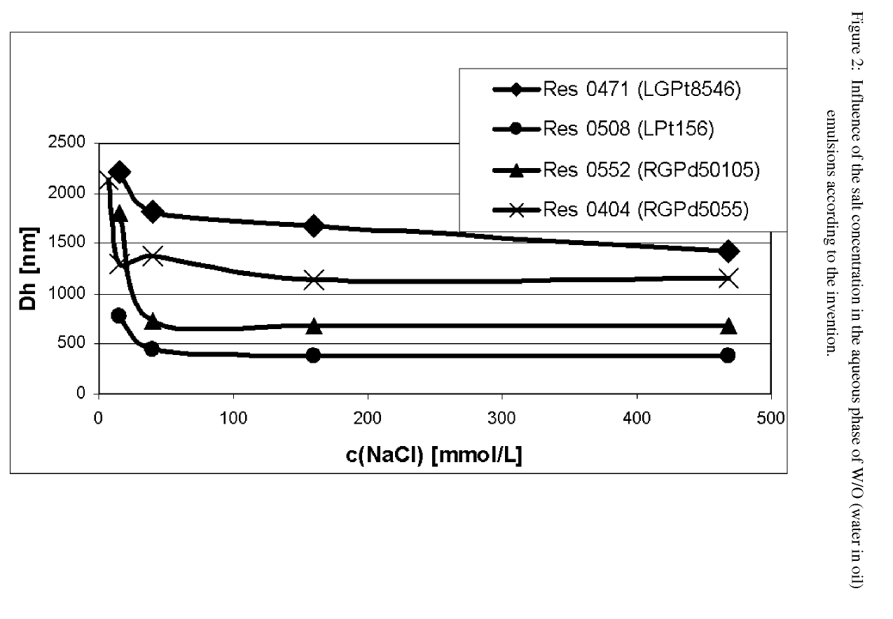

W/O (water in oil) emulsions which contain 4 different emulsifiers (see the information relating to FIG. 2) and similarly to Example 1 contain budesonide in the organic phase are shown in FIG. 2 (Res=Resomer®). In each case sodium chloride was dissolved in the inorganic phase which was used within the scope of the preparation of these W/O (water in oil) emulsions according to the invention. The concentrations equated to an ionic strength of 10 mM, at the minimum. When attempts were made to prepare comparable emulsions omitting the sodium chloride, it was observed that phase separation occurred spontaneously within less than 10 minutes after mixing of the phases, so that it was not possible to determine a hydrodynamic diameter/no stable emulsion was obtained (not shown, as no measured values can be obtained as a result of the instability).

### Stability of the Emulsion Droplet Size of the W/O (Water in Oil) Emulsions According to the Invention

### Example 3

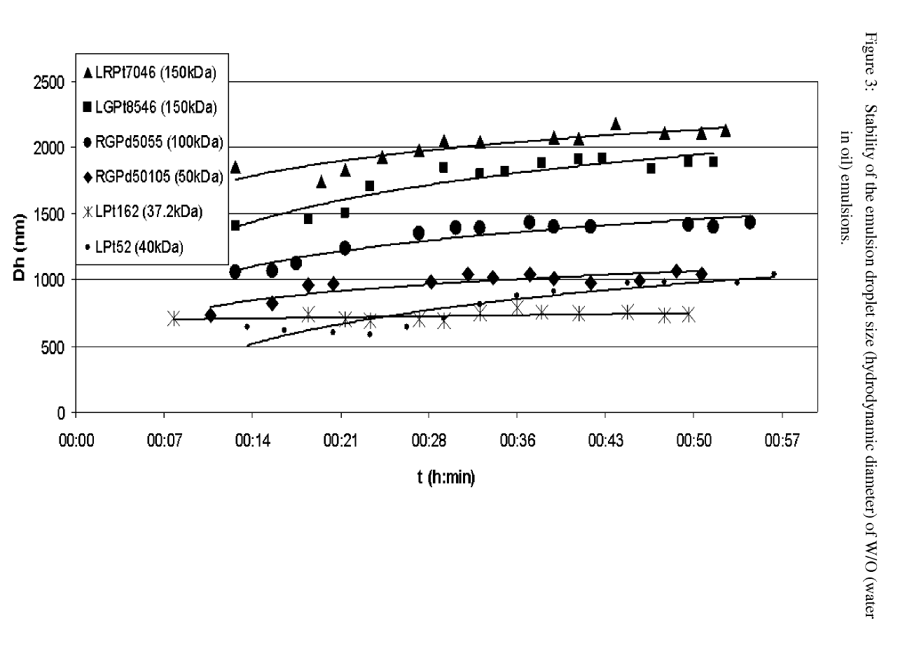

Examples of the stability characteristics of W/O (water in oil) emulsions containing an acid addition salt of an active substance, which is present in the aqueous phase in dissolved form, are shown in FIG. 3.

The W/O (water in oil) emulsions according to the invention with the results shown in FIG. 3 were obtained by dissolving various polymers of the Resomer® type as emulsifiers in the organic phase (DCM) (cf. the captions to the Figures and the information according to Table 1). Salbutamol sulphate salt was dissolved in the inorganic (aqueous) phase. 1: 4 W/O (water/DCM) emulsions were prepared from these phases by ultrasound treatment.

As can be seen in FIG. 3, all the W/O (water/DCM) emulsions according to the invention exhibit stable characteristics. The emulsion droplet size (hydrodynamic diameter of the emulsion) is in a suitable range between 300 nm and 3000 nm. Moreover the emulsion droplet size remains stably within this suitable range for the droplet size within the measuring period of 1 hour.

### Example 4

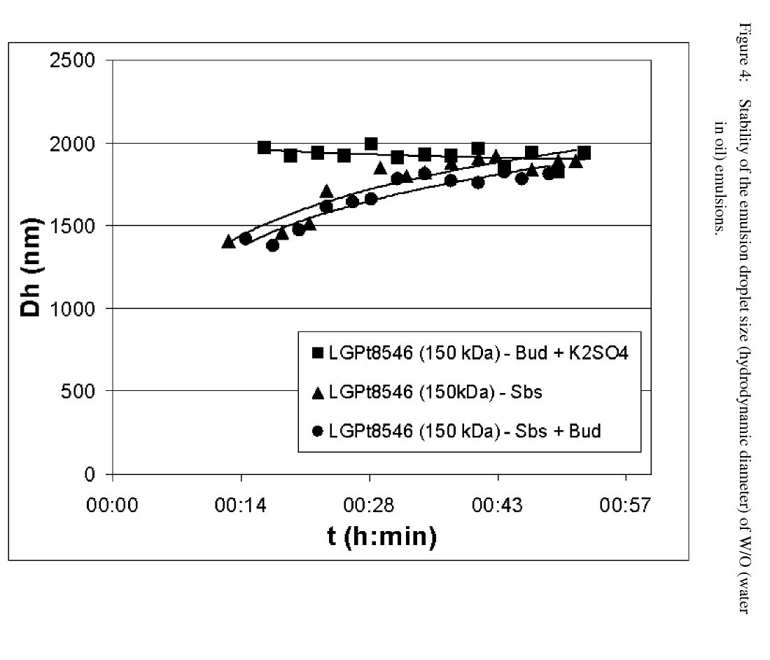

Further examples of the stability characteristics of W/O (water in oil) emulsions according to the invention are shown in FIG. 4. W/O (water in oil) emulsions according to the invention were prepared, analogously to Examples 1 and 3, by first dissolving potassium sulphate as the salt component in the aqueous phase (budesonide being dissolved in the organic phase, independently thereof), and also using salbutamol sulphate salt as the salt component in the aqueous phase (without budesonide in the organic phase) and in turn using salbutamol sulphate salt as the salt component in the aqueous phase but additionally dissolving budesonide in the organic phase. The emulsifier used in each case was LGPt8546 (see the captions to the Figure and information according to Table 1).

As can be seen from FIG. 4, all the W/O (water/DCM) emulsions according to the invention exhibit stable characteristics. The emulsion droplet size (hydrodynamic diameter of the emulsion) is in a suitable range between 300 nm and 3000 nm. Moreover the emulsion droplet size remains stably within this suitable range for the droplet size within the measuring period of 1 hour.

1. W/O (water in oil) emulsion which contains a pharmacologically active substance, characterised in that the emulsion droplets have a hydrodynamic diameter of between 200 nm and 3000 nm and the aqueous phase that is used to prepare this emulsion is characterised in that a salt is dissolved therein and the aqueous phase has an ionic strength of more than 10 m M.

2. W/O (water in oil) emulsion according to claim 1, characterised in that the salt dissolved in the aqueous phase is selected from among an alkali metal salt, an alkaline earth metal salt, an acid addition salt of an active substance or a combination thereof.

3. W/O (water in oil) emulsion according to claim 2, characterised in that besides a pharmacologically active substance an alkali metal salt or alkaline earth metal salt, preferably NaCl, is present in the aqueous phase.

4. W/O (water in oil) emulsion according to claim 1, characterised in that it contains as emulsifier a substance from the category of the block copolymers.

5. W/O (water in oil) emulsion according to claim 1, characterised in that it contains as emulsifier a substance from the group selected from the category of the PEG-[poly-(lactide-co-glycolides)] and the PEG-[poly-lactides].

6. W/O (water in oil) emulsion according to claim 5, characterised in that the emulsifier has a PEG content of between 1 to 15% (% by mass, based on the total molecular mass of the block copolymer), a molecular mass of 37.5 to 600 kD, a diblock or triblock structure, the glycolide content is between 0-50% (% be mass based on the poly-(lactide-co-glycolide) or the poly-lactide) and the lactide content is between 50-100% ((% by mass based on the poly-(lactide-co-glycolide) or the poly-lactide).

7. W/O (water in oil) emulsion according to claim 1, characterised in that a pharmacologically active substance is dissolved in the organic phase.

8. Process for preparing W/O (water in oil) emulsions according to claim 1, characterised in that
a. an organic phase which contains an emulsifier that represents a substance selected from among the category of the PEG-[poly-(lactide-co-glycolides)] and the category of the PEG-[poly-lactides],
b. an aqueous phase which contains a salt selected from among an alkali metal salt, an alkaline earth metal salt, an acid addition salt of a pharmacologically active substance or a combination thereof,
is incorporated by intensive mixing.

9. (canceled)
